(19) United States
(12) Patent Application Publication
(10) Pub. No.: US 2006/0241022 A1
(43) Pub. Date: Oct. 26, 2006

(54) Selectin targeting bioconjugates

(76) Inventors: Benjamin P. Bowen, Tempe, AZ (US); Stephen P. Massia, Phoenix, AZ (US); Gholam R. Ehteshami, Scottsdale, AZ (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/244,536

(22) Filed: Oct. 5, 2005

Related U.S. Application Data
(60) Provisional application No. 60/616,354, filed on Oct. 6, 2004.

(51) Int. Cl. A61K 38/17; C07K 14/74

(52) U.S. Cl. 514/8; 530/350

(57) ABSTRACT

The present invention provides novel compositions comprising one or more selectin binding molecule covalently linked to a hydrophilic polymer, pharmaceutical compositions thereof, and methods for their use in treating anti-inflammatory disorders.

### CROSS REFERENCE

This application claims the benefit of U.S. provisional patent application Ser. No. 60/616,354 filed Oct. 6, 2004, which is incorporated by reference herein in its entirety.

### STATEMENT OF GOVERNMENT INTEREST

The work herein was supported in part by NIH grant 1 K25 HL076381-01 A1; thus the United States Government may have certain rights to this invention.

### BACKGROUND OF THE INVENTION

The selectin family includes molecules that contain an N-terminal domain homologous to lectins. They are Ca²⁺ dependent transmembrane glycoproteins that bind to sialylated carbohydrate moieties present on target proteins. There are three different selectins: P-, E-, and L-selectin, and the type of cell on which it is predominantly expressed gives this naming convention (P=platelets; E=endothelial; L=leukocytes). The primary functions of selectins are lymphocyte homing and leukocyte recruitment to inflamed tissue.

E- and P-selectin expressed on the surface of endothelial cells loosely tether circulating leukocytes, which lead to leukocyte rolling along the vessel wall. Leukocyte rolling is crucial in bringing leukocytes to a site of inflammation where strong interactions involving ICAM-1 and other cell adhesion molecules anchor the cell prior to diapedesis. Recent experiments using molecules that specifically bind to selectins demonstrate that, in many cases, binding to selectins greatly reduces the inflammatory response. The physiology of selectins and their potential importance as a target for drug delivery is discussed in a recent review by Ehrhardt et. al. Adv. Drug Deliv. Rev. (2004) 56:527-549. While various selectin binding molecules have been made and tested for use as anti-inflammatory agents, all have shortcomings for therapeutic use. Thus, there is a need in the art for improved anti-inflammatory therapeutics that target one or more selecting.

### SUMMARY OF THE INVENTION

In one aspect, the present invention provides compositions comprising one or more selectin binding molecule covalently linked to a hydrophilic polymer. In a preferred embodiment, the composition comprises more than one selectin binding molecule, which can comprise multiple copies of the same selectin binding molecule, or more than one type of selectin binding molecule with the same or different selectin binding specificities. In various further preferred embodiments, the selectin binding molecule comprises a polypeptide and the hydrophilic polymer comprises a polysaccharide. In further preferred embodiments, the selectin binding molecules are attached alone or clustered at one terminus of the hydrophilic polymer.

In another aspect, the present invention provides pharmaceutical compositions comprising a composition of the invention and a pharmaceutically acceptable carrier.

In a further aspect, the present invention provides methods for treating inflammatory disorders, comprising administering to a patient in need thereof an amount effective to treat the inflammatory disorder of a composition according to the present invention.

### BRIEF DESCRIPTION OF THE FIGURES

FIG. 1. Two embodiments of bioconjugates of the invention where the polymer is shown to be bound to several selectin binding molecules (STMs).

FIG. 2. Schematic of a non-branched, end-only labeled dextran bioconjugate.

FIGS. 3A and B. Schematic of branched end-only dextran bioconjugate.

FIG. 4. Representative frames from video showing monocyte adhesion to inflamed endothelial cells (A), lack of adhesion to non-inflamed endothelial cells (B), and lack of adhesion to inflamed endothelial cells treated with e-selectin binding peptide-dextran conjugates (C).

FIG. 5. TNF-α treated Human aortic endothelial cells (HAECS) show little affinity for activated monocytes under physiological flow conditions when treated with e-selectin targeting dextran-peptide conjugates. At 880 nM the conjugate is able to effectively stop monocyte adhesion to inflamed endothelial cells at a shear stress of 1 dyne/cm². Samples not treated with peptide-dextran conjugate showed consistent monocyte rolling and adhesion on inflamed endothelial cells throughout the 60 seconds of data collected for each condition.

FIG. 6. Monocytes bound to TNF-α treated HAECS could be displaced with 1 mg/mL peptide-dextran conjugate. At time-zero, monocytes along with 1 mg/ml of conjugate were introduced to the flow chamber. Prior to time-zero, only activated monocytes were present in the flow chamber. For these conditions, 1 dyne/cm² shear stress was used. Shown for comparison is a separate dish of endothelial cells treated in the same way but without having bioconjugate in the solution.

### DETAILED DESCRIPTION OF THE INVENTION

In one aspect, the present invention provides compositions comprising a selectin binding molecule covalently linked to a hydrophilic polymer.

As used herein, the term “selectin binding molecule” means one or more molecules that bind directly to one or more of P-, E-, and L-selectin. Examples of such selectin binding molecules include, but are not limited to peptides, glycoproteins, antibodies, oligosaccharides, nucleic acid aptamers, and combinations thereof.

The selectin binding molecules for use in the invention preferentially bind to the one or more selectins in a mixture of molecules.

The selectin binding molecules can be presented on the surface of microspheres or beads, as noted in Table 1. However, when presented in this manner, the selectin binding molecules are still covalently bound to the hydrophilic polymer.

TABLE 1
Selectin Targeting molecules and references that correspond to those molecules.

| | |
|---|---|
| glycoprotein, which is sulfated, fucosylated, and sialylated | (1-8) |
| sialyl-Lewis x and -Lewis a (sLex and sLea, respectively) carbohydrate motifs | (1, 9-13) |
| synthetic oligosaccharides based on sialyl Lewis x | (14-17) |
| Monospecific Glycoprotein Ligands that bind E or P selectin preferentially | (18) |
| mucin-like P-selectin glycoprotein ligand 1 (PSGL-1) and peptides from it | (19-23) |
| Antibody that recognizes E-selectin | (24-30) |
| Homologous fucose sugar unit | (31) |
| beta-turn dipeptides | (31) |
| Antibody that recognizes P-Selectin | (20, 26, |
| | 30) |
| alpha m beta 2 integrin | (32) |
| E, P, and L Selectin Binding peptide IELLQAR (SEQ ID NO: 1) | (33, 34) |
| Peptide CDITWAQLWDLMK (SEQ ID NO: 2) | (35, 36) |
| Oligonucleotides specific for L-Selectin | (37) |
| Oligonucleotides specific for P-Selectin | (38) |
| Peptide KYDGDITWDQLWDLMK (SEQ ID NO: 3) that targets E-Selectin | (39) |
| peptide mimic of SA-Le(a) carbohydrate DLWDWVVGKPAG (SEQ ID | (40, 41) |
| NO: 4) based on the consensus sequence DXXDXXVG (SEQ ID NO: 5) | |
| Peptide containing the sequence EWVDV (SEQ ID NO: 6) that targets P- | (42) |
| Selectin | |
| P-Selectin targetting peptide from phage display | (43) |
| Antibody that recognizes PSGL-1 | (44) |

In a preferred embodiment, the selectin binding molecule comprises or consists of a polypeptide, oligosaccharide, or nucleic acid aptamer sequence that binds to one or more of the selectins. Such polypeptides, oligosaccharides, or nucleic acid aptamers may optionally be sialylated in order to promote stronger binding.

The term “polypeptide” is used in its broadest sense to refer to a sequence of subunit amino acids, amino acid analogs, or peptidomimetics. The subunits are linked by peptide bonds, except as noted. The polypeptides described herein may be naturally occurring, processed forms of naturally occurring polypeptides (such as by enzymatic digestion), chemically synthesized or recombinantly expressed. Recombinant expression can be accomplished using standard methods in the art, generally involving the cloning of nucleic acid sequences capable of directing the expression of the polypeptides in an expression vector, which can be used to transfect or transduce a host cell in order to provide the cellular machinery to carry out expression of the polypeptides. Such expression vectors can comprise bacterial or viral expression vectors, and such host cells can be prokaryotic or eukaryotic.

Preferably, the polypeptides for use in the methods of the present invention are chemically synthesized. Synthetic polypeptides, prepared using the well-known techniques of solid phase, liquid phase, or peptide condensation techniques, or any combination thereof, can include natural and unnatural amino acids. Amino acids used for peptide synthesis may be standard Boc (Nα-amino protected Nα-t-butyloxycarbonyl)amino acid resin with the standard deprotecting, neutralization, coupling and wash protocols of the original solid phase procedure of Merrifield, or the base-labile Nα-amino protected 9-fluorenylmethoxycarbonyl (Fmoc) amino acids first described by Carpino and Han. Both Fmoc and Boc Nα-amino protected amino acids can be obtained from Sigma, Cambridge Research Biochemical, or other chemical companies familiar to those skilled in the art. In addition, the polypeptides can be synthesized with other Nα-protecting groups that are familiar to those skilled in this art.

Solid phase peptide synthesis may be accomplished by techniques familiar to those in the art, or using automated synthesizers. The polypeptides of the invention may comprise D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, and norleucine for leucine or isoleucine.

In addition, the polypeptides can have peptidomimetic bonds, such as ester bonds, to prepare polypeptides with novel properties. For example, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo.

In exemplary embodiments, the selectin binding molecule comprises or consists of one or more repeats of a polypeptide (wherein “X” represents any amino acid residue) selected from the group consisting of:

| | |
|---|---|
| EWVDV; | (SEQ ID NO:6) |
| DLWDWVVGKPAG; | (SEQ ID NO:4) |
| DXXDXXVG; | (SEQ ID NO:5) |
| VVGXP; | (SEQ ID NO:7) |
| FVVGXP; | (SEQ ID NO:8) |
| DLWDFVVGKPAG; | (SEQ ID NO:9) |
| IELLQAR; | (SEQ ID NO:1) |
| LVSVLDLEPLDAAWL; | (SEQ ID NO:10) |
| KYDGDITWDQLWDLMK; | (SEQ ID NO:3) |
| CDITWAQLWDLMK; | (SEQ ID NO:2) |
| and | |
| QATEYEYLDYDFLPETEPP. | (SEQ ID NO:11) |

The oligosaccharide can be related to the tetrasaccharide Sialyl Lewis X structure that is present on the surface of white blood cells. Exemplary oligosaccharides comprise or consist of one or more sLex (sialyl Lewis x oligosaccharide) and/or sLea (sialyl Lewis a oligosaccharide) carbohydrate motifs (1, 9-13).

In one embodiment, the selectin binding molecule binds to E selectin, such as selectin binding molecules in the following Table:

| | |
|---|---|
| Antibody that recognizes E-selectin | (24-30) |
| Peptide KYDGDITWDQLWDLMK (SEQ ID NO:3) | (39) |
| that targets E-Selectin | |

Thus, in one embodiment wherein the selectin binding molecule binds to E selectin, the selectin binding molecule comprises or consists of one or more repeats of a polypeptide of the amino acid sequence KYDGDITWDQLWDLMK (SEQ ID NO:3). In a further embodiment, the selectin binding molecule comprises or consists of an antibody that selectively binds to E selectin, such as those described in references 24-30, or any other antibody selective for E selectin.

In another embodiment, the selectin binding molecule binds to P selectin

| | |
|---|---|
| mucin-like P-selectin glycoprotein ligand 1 (PSGL-1) and | (19-23) |
| peptides from it | |
| Antibody that recognizes P-Selectin | (20, 26, |
| | 30) |
| Oligonucleotides specific for P-Selectin | (38) |
| Peptide containing the sequence EWVDV (SEQ ID NO: 6) | (42) |
| that targets P-Selectin | |
| P-Selectin targetting peptide from phage display | (43) |

Thus, in one embodiment wherein the selectin binding molecule binds to P selectin, the selectin binding molecule comprises or consists of one or more repeats of a polypeptide of the amino acid sequence EWVDV (SEQ ID NO:6). In a further preferred embodiment, the selectin binding molecule comprises or consists of an antibody that selectively binds to P selectin.

In a further embodiment, the selectin binding molecule binds to L selectin, such as:

| | |
|---|---|
| Oligonucleotides specific for L-Selectin | (37) |

In a further preferred embodiment, the selectin binding molecule comprises or consists of an antibody that selectively binds to L selectin.

In further embodiments, the selectin binding molecule binds to two or more of the selectins, such as those shown in the Table below:

| | |
|---|---|
| Monospecific Glycoprotein Ligands that bind E or P | (18) |
| selectin preferentially | |
| sialyl-Lewis x and -Lewis a (sLex and sLea, respectively) | (1, 9-13) |
| carbohydrate motifs | |
| synthetic oligosaccharides based on sialyl Lewis x | (14-17) |
| E, P, and L Selectin Binding peptide IELLQAR | (33, 34) |
| (SEQ ID NO: 1) | |

In an example of this embodiment, the selectin binding molecule comprises or consists of a polypeptide of an amino acid sequence comprising or consisting of one or more repeats of IELLQAR (SEQ ID NO:1). In a further preferred embodiment, the selectin binding molecule comprises or consists of an antibody that selectively binds to two or more of selectins E, P, and L.

As noted above, the hydrophilic polymer carries one or more selectin binding molecules that are covalently bound to the polymer; such compositions are also referred to as “selectin binding bioconjugates.” The conjugation of the selectin binding molecules to the hydrophilic polymers serves to provide one or more benefits compared to the selectin binding molecule alone. For example, multivalent presentation (i.e.: more than one selectin binding molecule) of the selectin binding molecules increases the amount of time that the bioconjugate is bound to the target; the covalent bond to the hydrophilic polymer can inhibit the breakdown of the selectin targeting molecule; and the hydrophilic polymer acts to block cells and molecules from accessing the selectin target.

The various selectin binding molecules disclosed above can be used alone, wherein a composition according to the invention contained multiple copies of the single selectin binding molecule, or the composition can comprise different selectin binding molecules, with the same or differing specificities, depending on the purpose for which the composition is to be used.

Antibodies for use in the present invention can be made by well-known methods, such as described in Harlow and Lane, Antibodies; A Laboratory Manual, Cold Spring Harbor Laboratory, Cold Spring Harbor, N.Y., (1988), and Kohler and Milstein, Nature 256, 495-497 (1975). Such antibodies can be polyclonal or monoclonal, and monoclonal antibodies can be either partially or fully humanized. “Humanized antibody” refers to antibodies derived from a non-human antibody, such as a mouse monoclonal antibody. Alternatively, humanized antibodies can be derived from chimeric antibodies that retain or substantially retain the antigen-binding properties of the parental, non-human, antibody but which exhibit diminished immunogenicity as compared to the parental antibody when administered to humans. For example, chimeric antibodies can comprise human and murine antibody fragments, generally human constant and mouse variable regions. Since humanized antibodies are far less immunogenic in humans than the non-human monoclonal antibodies, they are preferred for therapeutic antibody use. The term antibody as used herein is intended to include antibody fragments thereof which are selectively reactive with P, E, or L selectin, or fragments thereof. Antibodies can be fragmented using conventional techniques, and the fragments screened for selectin binding using standard methods in the art. For example, F(ab′)₂ fragments can be generated by treating antibody with pepsin. The resulting F(ab′)₂ fragment can be treated to reduce disulfide bridges to produce Fab′ fragments.

As used herein the term “hydrophilic polymer” refers to polymers that are soluble in aqueous solutions, and wherein the polymer backbone is not itself a selectin binding molecule. In a preferred embodiment, the polymer is soluble at a concentration of 1 mg of polymer per ml of aqueous solution. In a further preferred embodiment, the polymer limits cell adhesion, to provide a better barrier for the methods of the invention, as discussed below.

In a further preferred embodiment, the hydrophilic polymer comprises or consists of naturally occurring or synthetic polysaccharides, which provides multivalent covalent binding for the selectin binding molecule. In this embodiment, it will be apparent to those of skill in the art that the selectin binding molecule may comprise multiple such binding molecules, in order to present different selectin binding molecules in a single composition. In a preferred multivalent embodiment, between 1% and 30% of the residues available on the polysaccharide for binding the selectin binding molecule are covalently bound to selectin binding molecules; more preferably between 2%-30%, 3%-30%, 4%-30%, 5%-30%, 10%-30%, 15%-30%, 20%-30%, 25%-30%, 5%-25%, 5%-20%, 5%-15%, 5%-10%, 10%-25%, 10%-20%, 10%-15%, 15%-25%, or 15%-20%. In a most preferred embodiment, the polysaccharide is dextran, and the valency of selectin binding molecule attachment on the glucopyranose residues of dextran is as above.

For example, the composition could comprise multiple selectin binding molecules that bind to different portions of a single selectin. Alternatively, the composition could comprise different selectin binding molecules that bind to different members of the selectin family. Many such permutations will be readily apparent to those of skill in the art based on the teachings of the present invention.

Oligosaccharides, polysaccharides, or derivatives thereof (derived from microbial species, prokaryotic or eukaryotic organisms, or chemical synthesis) may also serve as hydrophilic polymers. Non-limiting examples of polysaccharides for use as polymers in the present invention are polynucleotides, starch, cellulose, chitin, agarose, dextran, heparin, chondroitin sulfate, hyaluronic acid, and hydroxyethyl starch.

Further non-limiting examples of appropriate hydrophilic polymers for use in the present invention include but are not limited to polyamides, poly(ethylene glycol), poly(ethylene oxide), poly(vinyl alcohol), poly(acrylic acid), poly(ethylene-co-vinyl alcohol), poly(vinyl pyrrolidone), poly(ethyloxazoline), and poly(ethylene oxide)-co-poly(prophylene oxide) block copolymers. In addition, the hydrophilic polymer can comprise copolymers, block copolymers (which can be modified with blocks polymerized on one or more ends), graft copolymers, alternating polymers, random polymers, and/or branched polymers.

As used herein, a co-polymer is a polymer composed of 2 or more different monomer units.

As used herein, a block copolymer is a polymer composed of linear segments containing 1 or more monomers of the same type, which are covalently attached to at least one other segment containing one or more monomers of a different type. Exemplary blocks to be polymerized at the block polymer ends can be composed of lactic acid, glycolic acid, e-caprolactone, lactic-co-glycolic acid oligomers, trimethylene carbonate, anhydrides, and amino acids. This list is not exhaustive; other oligomers may also be used for block copolymers.

As used herein, a graft copolymer comprises one or more polymer chain to which are covalently attached, along their backbone, one or more linear or branched chains containing one or more monomer unit.

As used herein, an alternating copolymer comprises polymer chains containing either alternating monomers of a different type or alternating blocks of monomers of different type.

As used herein, a random copolymer comprises two or more monomer units that do not occur along the backbone in an alternating fashion.

As used herein, a branched polymer has a non-linear arrangement of monomers. Examples of such branched polymers include polyethylene glycol (PEG) star polymers, PEG comb polymers, lysine dendrimers, and other dendrimers.

The hydrophilic polymer can also be a solid, such as a colloidal particle. Serum proteins such as albumin could also serve as a low cell-adhesive colloidal component.

In a still further preferred embodiment or each embodiment disclosed herein, the compositions of the invention do not include any lipid component.

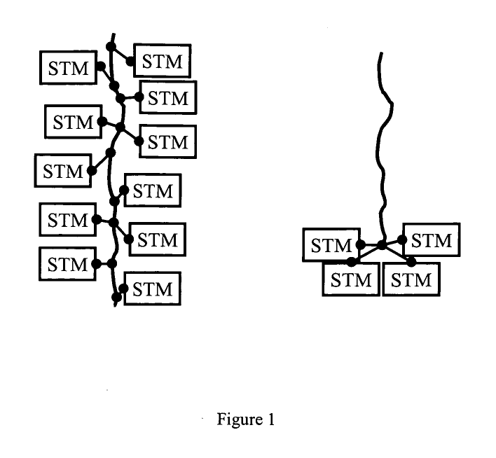

The selectin binding molecules are covalently bound to the hydrophilic polymer using standard methods in the art. Examples of such methods are discussed in the examples below. FIG. 1 shows two structurally different embodiments of the compositions of the invention.

The first structural embodiment has selectin targeting molecules distributed throughout a polymer. This is preferred when even distribution of the selectin binding molecule(s) over the entire polymer is desired, for example, when E-selectin is targeted and the bioconjugate is expected to bind preferentially to endothelial cells. This embodiment is especially applicable towards creating a barrier-coating on epithelial tissue to treat or prevent pathological inflammation.

Such distribution of the selectin targeting molecule on the polymer can be achieved using standard techniques in the art. For example, the polymer can be derivatized to provide covalent binding sites for the selectin targeting molecule of interest.

The second structural embodiment shown in FIG. 1 has a cluster of selectin targeting molecules at one end. This embodiment may lead to improved performance, and is especially preferred when a less dense overall distribution of the selectin binding molecule on the polymer is desired, for example, when platelets are the preferential target of the bioconjugate and it is desired to limit the possibility of aggregation. In addition to limiting aggregation of circulating cells, it is proposed that high-molecular weight colloids labeled only at one terminus with a selectin targeting molecule form a surface on the cell which they are bound to that under certain conditions will be better able to limit the binding of other cells. This embodiment is particularly useful for creating a mimic of the glycocalyx which is often lost during an inflammatory response. The structural difference between the two embodiments is the length of unmodified polysaccharide and the number/density of selectin targeting molecules on the bioconjugate.

Such distribution of the selectin targeting molecule on the polymer can be achieved using standard techniques in the art, as well as the methods described below

Solid-Phase Synthesis of End-Only Labeled Dextran

Following the work of Zhao et. al. (1997), end-only peptide-dextran conjugates are synthesized. A schematic depicting this structure is shown in FIG. 1B. Initially, a solid-phase synthesis protocol is developed for making end-only-labeled peptide dextran conjugates. This differs from the work of Zhao et. al. in that purification of unreacted dextran is easily done compared to gel-based separations that are required for solution phase chemistry.

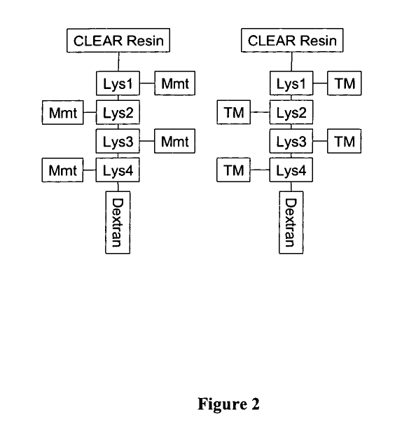

Initially, one or more lysine residues are synthesized on a CLEAR™ column (Cross-Linked Ethoxylate Acrylate Resin, Peptides International, Inc.) with standard Fmoc peptide synthesis. The epsilon-amines are protected with 4-methoxytrityl (mMt) from NovaBiochem. Onto the terminal amine, the dextran is conjugated by reductive amination by circulating dextran with NaCNBH₃ through the column in pH 8 buffer for 24 hours. This can bee seen in FIG. 2. The CLEAR™ column has been shown to have acceptable swelling characteristics in aqueous solution. Following deprotection of the mMt group on the epsilon amines, the selectin-binding molecule (TM) is covalently attached to the ε-amine of the lysine residues by its carboxy terminus after activation. The entire bioconjugate is washed and cleaved from the column using standard methods. Purification by size-exclusion chromatography provides an estimate of the yield and remove any peptide material that never reacted with dextran.

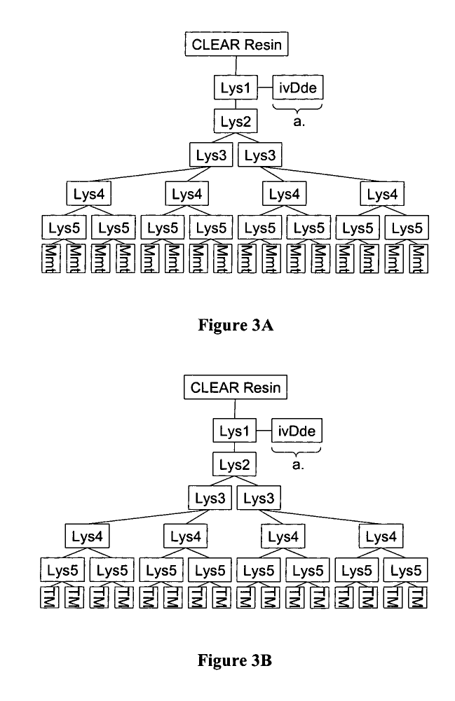

In another embodiment of this proposed work, a lysine-dendrimer containing 2, 4, 8, or 16 amines is synthesized. The final layer/generation of lysine amines have all primary amines protected with an mMt-protecting group. The epsilon-amine on the first lysine residue added is protected by an 1-(4,4-Dimethyl-2,6-dioxo-cyclohexylidene)-3-methylbutyl (ivDde) group. This enables 2 separate deprotection steps (Mmt and ivDde) so that a single amine is available after treatment with hydrazine allowing the coupling of a single dextran molecule to the first lysine residue, and treatment with 1% TFA frees the amines on the final generation of lysine residues allowing attachment of targeting peptides. This embodiment is depicted in FIG. 3. The entire bioconjugate is washed and cleaved from the column using standard methods. Purification by size-exclusion chromatography provides an estimate of the yield and removes any peptide material that never reacted with dextran.

In a further embodiment, the synthesis described by Zhao is performed in solution using the purification methods described in his work. A multivalent targeting molecule is synthesized on a column where the starting resin contains a branched poly-lysine dendritic structure. Multivalent molecules containing from 1 to 16 targetting peptides are purified and end-only coupled to dextran by a single primary amine in the same manner described by previous work.

The compositions of the invention may be subjected to conventional pharmaceutical operations such as sterilization and/or may contain conventional adjuvants, such as preservatives, stabilizers, wetting agents, emulsifiers, buffers etc. Thus, in another aspect, the present invention provides pharmaceutical compositions, comprising the compositions of the invention and a pharmaceutically acceptable carrier. For administration, the compositions are ordinarily combined with one or more adjuvants appropriate for the indicated route of administration. The compositions may be admixed, for example, with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, dextran sulfate, heparin-containing gels, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The compositions may be administered by any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. In a most preferred embodiment, the compositions are administered intravenously.

The compositions may be made up in a solid form (including granules, powders or suppositories) or in a liquid form (e.g., solutions, suspensions, or emulsions). The polypeptides of the invention may be applied in a variety of solutions. Suitable solutions for use in accordance with the invention are sterile, dissolve sufficient amounts of the compositions, and are not harmful for the proposed application.

The compositions may also be included with other compositions to provide additional benefit. In one embodiment, the compositions are included in plasma extenders, which are solutions used to extend or increase the amount of blood plasma in a patient in need thereof, such as after a trauma. In another embodiment, the compositions can be added to a wound dressing for use with, for example, burn patients.

In another aspect, the present invention provides methods for treating inflammatory disorders comprising administering to a patient in need thereof an amount effective to treat the inflammatory condition of one or more composition according to the present invention. The compositions of the invention serve as a class of anti-inflammatory/immuno-suppressant therapeutics that selectively target and locally bind to inflamed tissue surfaces forming a protective colloid barrier against pathologically driven excessive leukocyte adhesions/infiltration and subsequent tissue injury. Although leukocyte adhesion to tissue surfaces is essential for normal immune system function, leukocyte/tissue adhesion plays a major role in a number of pathological processes including septic shock, post-trauma multiple organ failure, ischemic reperfusion injury, transplant rejection, inflammatory diseases, autoimmune diseases, rheumatoid arthritis, psoriasis, inflammatory bowel disease, adult respiratory distress syndrome, tumor metastasis, and burns. Therefore the compositions can be used as a therapy delivered throughout the vasculature that selectively and locally targets leukocyte-adhesive tissues to suppress pathologically excessive leukocyte-mediated damage to healthy tissues and thus limit deleterious outcomes. Thus, in this embodiment, the methods may be used to treat one or more of septic shock, post-trauma multiple organ failure, ischemic reperfusion injury, transplant rejection, inflammatory diseases, autoimmune diseases, rheumatoid arthritis, psoriasis, inflammatory bowel disease, adult respiratory distress syndrome, tumor metastasis, and burns.

As used herein, “treat” or “treating” means accomplishing one or more of the following: (a) reducing the severity of the disorder; (b) limiting or preventing development of symptoms or complications characteristic of the disorder(s) being treated; (c) inhibiting worsening of symptoms or complications characteristic of the disorder(s) being treated; (d) limiting or preventing recurrence of the disorder(s) in patients that have previously had the disorder(s); and (e) limiting or preventing recurrence of symptoms or complications in patients that were previously symptomatic for the disorder(s).

As used herein, an “amount effective” of the one or more compositions is an amount that is sufficient to provide the intended benefit of treatment. An effective amount of the bioconjugate that can be employed ranges generally between about 0.01 μg/kg body weight and about 10 mg/kg body weight, preferably ranging between about 0.05 μg/kg and about 5 mg/kg body weight. However dosage levels are based on a variety of factors, including the type of injury, the age, weight, sex, medical condition of the individual, the severity of the condition, the route of administration, and the particular compound employed. Thus, the dosage regimen may vary widely, but can be determined routinely by a physician using standard methods

The compositions can be administered as the sole therapeutic agent, or can be combined with other therapeutics known to be useful in treating inflammatory disorders, including but not limited to steroidal anti-inflammatories, non-steroidal anti-inflammatories, tumor necrosis factor, and COX-2 inhibitors.

In another aspect, the present invention provides methods for making the compositions of the invention, according to the methods disclosed below, and equivalents thereof.

### EXAMPLES

Synthesis

It will be understood by those of skill in the art that the following synthetic methods are examples of such methods, and are not meant to limit the methods that can be used to produce the bioconjugates of the invention.

In one example, dextran is oxidized to produce aldehyde groups via standard periodate methods (Wilson, M. B. and Nakane, P. K. Covalent coupling of proteins to periodate-oxidized Sephadex—new approach to immunoadsorbent preparation. Journal Of Immunological Methods. 12: 171-181, 1976). Dextran (M.W. 40 kDa, Sigma) is first dissolved in deionized water. Sodium periodate (NaIO₄, 0.1M) is prepared for immediate use. This NaIO₄ solution is added to the solution of dextran to make a 50% molar ratio of NaIO₄ to dextran (moles of glucose monomer). The reaction mixture is stirred at 4° C. overnight and protected from light by covering the reaction flask with aluminum foil. The solution is purified by precipitation of unreacted periodate and iodate products using an equimolar aqueous solution of BaCl₂. The purified oxidized dextran solution is lyophilized and stored (if not immediately used) at 4° C. in a 50 mL conical centrifuge tube protected from light. The product is analyzed by FTIR to demonstrate a peak at 1700 nm indicating the aldehyde groups within the dextran chain, evidence that oxidation occurred.

In this example, the selectin binding molecule should contain one or more terminal amines. A 10% stoichiometric excess of peptide is mixed in phosphate buffer (pH 8.0) for 48 hours at room temperature to conjugate the peptide to all of the aldehyde groups on the dextran. To form a more stable bond, the Schiff's base is reduced with sodium borohydride. Purification is accomplished by overnight dialysis or size-exclusion chromatography. The sample is lyophilized and stored at 4° C.

In another example, a selectin binding molecule containing a terminal thiol (SH) group is reacted with methacroylated dextran, which is synthesized using methods described by van Dijk-Wolthuis et al (van Dijk-Wolthuis, W N E, Franssen, O., Talsma, H., van Steenbergen, M. J., Kettenes-van den Bosch, J. J., and Hennink, W. E., Synthesis, characterization and polymerization of glycidyl methacrylate derivatized dextran, Macromolecules (1998), 28: 6317-6322.). Dextran (MW 70 kD) and dimethylaminopyridinine (DMAP) are dissolved in dimethylsulfoxide (DMSO) under nitrogen atmosphere at room temperature. Glycidyl methacrylate (GMA) is added to the mixture to produce GMA-derivatized dextran (dex-GMA). The amount of GMA is adjusted to obtain a degree of substitution (DS: molar ratio of GMA per glucopyranose residue) of 10. The reaction is terminated after 48 hours. The product is purified from the reaction mixture by solvent removal and size exclusion chromatography. Aqueous solutions of methacroylated dextran are rapidly frozen in liquid nitrogen, lyophilized, and stored frozen. The degree of substitution in purified product is determined by NMR.

Terminal thiol containing selectin binding molecules are added to phosphate buffered saline (PBS) with 1.5 mM EDTA. The pH is adjusted to 8.0-8.5 with triethanolamine (TEA). Methacroylated dextran will then be added to the reaction mix and the pH will be adjusted again to pH 8.0-8.5 with TEA. All solutions are maintained under inert conditions to minimize disulfide bond formation. The reaction is allowed to proceed at room temperature for 2 hours. The reaction mixture is dialyzed against deionized water in 25,000 MWCO membrane to remove any unreacted or disulfide bonded peptide. The purified dextran/peptide conjugates is recovered by lyophilization.

Inflammatory Cell Adhesion Assays

An in vitro inflammatory cell adhesion assay was used to determine the biological activity of dextran/E-Selectin binding peptide conjugates. An anti-inflammatory dextran/polypeptide bioconjugate was synthesized by coupling a synthetic polypeptide (CDITWAQLWDLMK) (SEQ ID NO:2) based on the work of Martens et. al. J. Biol. Chem., (1995) 270:21129-21136, according to the following methods. Synthetic peptides having the sequence described above were added to phosphate buffered saline (PBS) with 1.5 mM EDTA at a final concentration of 20 mM. The pH was adjusted to 8.0-8.5 with triethanolamine (TEA). Methacrylated dextran (2 mM) was then added to the reaction mix and the pH was adjusted again to pH 8.0-8.5. All solutions were maintained under inert conditions to minimize disulfide bond formation. Conjugation was allowed to proceed at room temperature for 2 hours. The reaction mixture was then dialyzed against deionized water in 10,000 MW cut-off membrane to remove any unreacted or disulfide bonded peptide. The purified dextran/peptide conjugates was recovered by lyophilization. In a similar manner, a dextran/polypeptide bioconjugate was made using the polypeptide (CDITWDQLWDLMK) (SEQ ID NO:12).

To assess the effect of these polypeptide-dextran bioconjugates on inflammatory cell adhesion, the following in vitro selectin-mediated leukocyte cell adhesion assay was performed. Human aortic endothelial cell (HAEC) monolayers were established on 35 mm plastic tissue culture dishes. In order to establish inflammation in one of the treatment groups, at 4 hours prior to the assay, normal culture medium was replaced with medium containing tumor necrosis factor α (10 ng/ml TNF-α). Following the incubation period, each sample was placed in a flow chamber containing media either with or without peptide-dextran conjugate. Treated sample groups received medium containing 1 mg/mL dextran-peptide conjugate (dextran conjugated to the peptide CDITWAQLWDLMK) (SEQ ID NO:12). Each dextran molecule contains approximately 64 peptide molecules. Untreated control samples received normal medium. For all treatment groups, flow chamber medium containing WEHI monocytic cells activated by 5 minute exposure to 50 nM Phorbol 12-Myristate 13-Actetate (PMA) (0.5-1×10⁵ cells/ml) were used. All samples were then incubated for another 2-3 minutes at physiological flow rates. Video data was recorded for 1-minute for each sample.

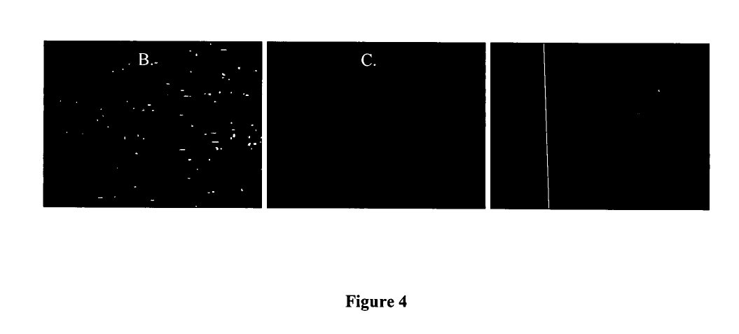

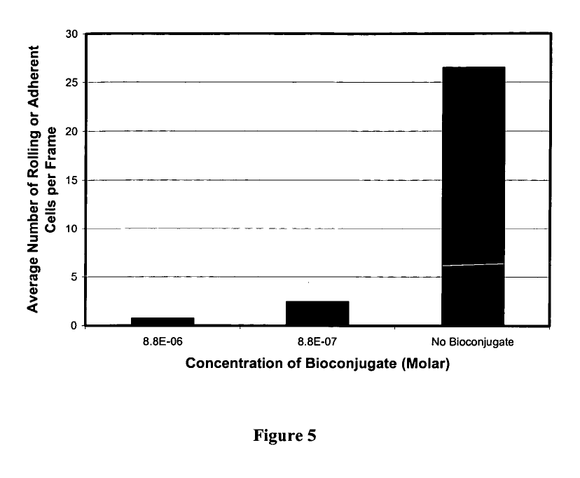

As can be seen in FIGS. 4 and 5, endothelial cells treated with TNF-α showed continued adhesion to the WEHI monocytic cells during the entire observation period; whereas the endothelial cells treated with TNF-α and 880 nM peptide-dextran conjugate did not show adhesion to monocytic cells during the observation period. The amount of adhesion to the endothelial cells treated with polypeptide-dextran conjugate was comparable to that observed for endothelial cells never exposed to TNF-α.

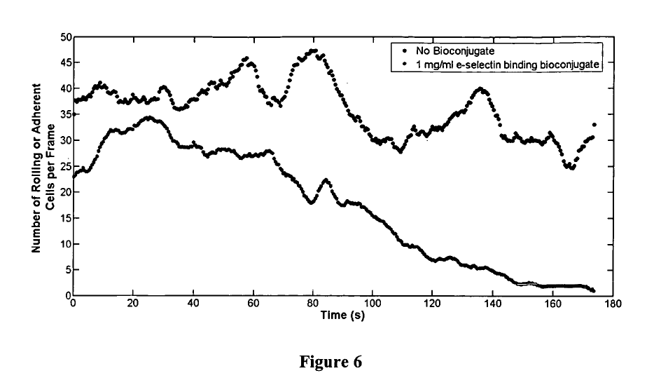

A separate experiment was performed where the peptide-dextran conjugate was used to displace bound WEHI monocytes. This result can be seen in FIG. 3. Monocytes were introduced into the flow chamber. Following this, monocytes with 1 mg/ml peptide-dextran conjugate was added. As can be seen in FIG. 6, all bound monocytes were removed after 150 seconds. This experiment was performed at a shear stress of 1 dyne/cm².

These experiments demonstrate that the compositions of the invention can dramatically reduce monocyte adhesion to endothelial cells under flow and shear stress conditions similar to that in the blood stream. Such activity makes this class of compounds especially useful in anti-inflammatory/immuno-suppressant therapeutics that selectively target and locally bind to inflamed tissue surfaces forming a protective colloid barrier against pathologically driven excessive leukocyte adhesions/infiltration and subsequent tissue injury.

In another bioconjugate version, the dextran/SEQ ID NO:2 bioconjugate was made but included a short linker of 6 ethoxy groups. The linker has an amine terminus and it was attached to the amine-end of the polypeptide by an amide bond. This linker can be used, for example, to couple polypeptides components of the bioconjugate to oxidized dextran.

### REFERENCES

1. Paavonen, T., and Renkonen, R. (1992) American Journal of Pathology 141, 1259-1264.
2. Imai, Y., Lasky, L. A., and Rosen, S. D. (1992) Glycobiology 2, 373-381.
3. Imai, Y., Lasky, L. A., and Rosen, S. D. (1993) Nature 361, 555-557.
4. Rosen, S. D. (1993) Histochemistry 100, 185-191.
5. Crommie, D., and Rosen, S. D. (1995) Journal of Biological Chemistry 270, 22614-22624.
6. Bruehl, R. E., Bertozzi, C. R., and Rosen, S. D. (2000) Journal of Biological Chemistry 275, 32642-32648.
7. Li, L., Short, H. J., Qian, K. X., Elhammer, A. P., and Geng, J. G. (2001) Biochemical and Biophysical Research Communications 288, 637-644.
8. Kanamori, A., Kojima, N., Uchimura, K., Muramatsu, T., Tamatani, T., Berndt, M. C., Kansas, G. S., and Kannagi, R. (2002) Journal of Biological Chemistry 277, 32578-32586.
9. Mebius, R. E., and Watson, S. R. (1993) Journal of Immunology 151, 3252-3260.
10. Yoshino, K., Ohmoto, H., Kondo, N., Tsujishita, H., Hiramatsu, Y., Inoue, Y., and Kondo, H. (1997) Journal of Medicinal Chemistry 40, 455-462.
11. Barbaro, J. (2004) Current Organic Chemistry 8, 883-902.
12. Eniola, A. O., and Hammer, D. A. (2003) Journal of Controlled Release 87, 15-22.
13. Ali, M., Hicks, A. E. R., Hellewell, P. G., Thoma, G., and Norman, K. E. (2003) Faseb Journal 17.
14. Nelson, R. M., Dolich, S., Aruffo, A., Cecconi, O., and Bevilacqua, M. P. (1993) Journal of Clinical Investigation 91, 1157-1166.
15. Skurk, C., Buerke, M., Guo, J. P., Paulson, J., and Lefer, A. M. (1994) American Journal of Physiology-Heart and Circulatory Physiology 36, H2124-H2131.
16. Hiramatsu, Y., Tsukida, T., Nakai, Y., Inoue, Y., and Kondo, H. (2000) Journal of Medicinal Chemistry 43, 1476-1483.
17. Bowman, K. G., Cook, B. N., de Graffenried, C. L., and Bertozzi, C. R. (2001) Biochemistry 40, 5382-5391.
18. Lenter, M., Levinovitz, A., Isenmann, S., and Vestweber, D. (1994) Journal of Cell Biology 125, 471-481.
19. Spertini, O., Cordey, A. S., Monai, N., Giuffre, L., and Schapira, M. (1996) Journal of Cell Biology 135, 523-531.
20. Kaytes, P. S., and Geng, J. G. (1998) Biochemistry 37, 10514-10521.
21. Norman, K. E., Katopodis, A. G., Thoma, G., Kolbinger, F., Hicks, A. E., Cotter, M. J., Pockley, A. G., and Hellewell, P. G. (2000) Blood 96, 3585-3591.
22. Burch, E. E., Patil, V. R. S., Camphausen, R. T., Kiani, M. F., and Goetz, D. J. (2002) Blood 100, 531-538.
23. Xia, L. J., Ramachandran, V., McDaniel, J. M., Nguyen, K. N., Cummings, R. D., and McEver, R. P. (2003) Blood 101, 552-559.
24. Spragg, D. D., Alford, D. R., Greferath, R., Larsen, C. E., Lee, K. D., Gurtner, G. C., Cybulsky, M. I., Tosi, P. F., Nicolau, C., and Gimbrone, M. A. (1997) Proceedings of the National Academy of Sciences of the United States of America 94, 8795-8800.
25. Harari, O. A., Wickham, T. J., Stocker, C. J., Kovesdi, I., Segal, D. M., Huehns, T. Y., Sarraf, C., and Haskard, D. O. (1999) Gene Therapy 6, 801-807.
26. Blackwell, J. E., Dagia, N. M., Dickerson, J. B., Berg, E. L., and Goetz, D. J. (2001) Annals of Biomedical Engineering 29, 523-533.
27. Everts, M., Kok, R. J., Asgeirsdottir, S. A., Melgert, B. N., Moolenaar, T. J. M., Koning, G. A., van Luyn, M. J. A., Meijer, D. K. F., and Molema, G. (2002) Journal of Immunology 168, 883-889.
28. Asgeirsdottir, S. A., Kok, R. J., Everts, M., Meijer, D. K. F., and Molema, G. (2003) Biochemical Pharmacology 65, 1729-1739.
29. Bhaskar, V., Law, D. A., Ibsen, E., Breinberg, D., Cass, K. M., DuBridge, R. B., Evangelista, F., Henshall, S. M., Hevezi, P., Miller, J. C., Pong, M., Powers, R., Senter, P., Stockett, D., Sutherland, R. L., von Freeden-Jeffry, U., Willhite, D., Murray, R., Afar, D. E. H., and Ramakrishnan, V. (2003) Cancer Research 63, 6387-6394.
30. Yun, Y. H., Goetz, D. J., Yellen, P., and Chen, W. L. (2004) Biomaterials 25, 147-157.
31. Hiramatsu, Y., Moriyama, H., Kiyoi, T., Tsukida, T., Inoue, Y., and Kondo, H. (1998) Journal of Medicinal Chemistry 41, 2302-2307.
32. Crutchfield, K. L., Patil, V. R. S., Campbell, C. J., Parkos, C. A., Allport, J. R., and Goetz, D. J. (2000) Journal of Leukocyte Biology 67, 196-205.
33. Fukuda, M. N., Ohyama, C., Lowitz, K., Matsuo, O., Pasqualini, R., Ruoslahti, E., and Fukuda, M. (2000) Cancer Research 60, 450-456.
34. Renkonen, R., Fukuda, M. N., Petrov, L., Paavonen, T., Renkonen, J., Hayry, P., and Fukuda, M. (2002) Transplantation 74, 2-6.
35. Egbrink, M., Janssen, G., Ookawa, K., Slaaf, D. W., Reneman, R. S., Wehrens, X. H. T., Maaijwee, K. J. M., Ohshima, N., Boudier, H., and Tangelder, G. J. (2002) Journal of Investigative Dermatology 118, 323-326.
36. Martens, C. L., Cwirla, S. E., Lee, R. Y. W., Whitehorn, E., Chen, E. Y. F., Bakker, A., Martin, E. L., Wagstrom, C., Gopalan, P., Smith, C. W., Tate, E., Koller, K. J., Schatz, P. J., Dower, W. J., and Barrett, R. W. (1995) Journal of Biological Chemistry 270, 21129-21136.
37. Oconnell, D., Koenig, A., Jennings, S., Hicke, B., Han, H. L., Fitzwater, T., Chang, Y. F., Varki, N., Parma, D., and Varki, A. (1996) Proceedings of the National Academy of Sciences of the United States of America 93, 5883-5887.
38. Jenison, R. D., Jennings, S. D., Walker, D. W., Bargatze, R. F., and Parma, D. (1998) Antisense & Nucleic Acid Drug Development 8, 265-279.
39. Zinn, K. R., Chaudhuri, T. R., Smyth, C. A., Wu, Q., Liu, H. G., Fleck, M., Mountz, J. D., and Mountz, J. M. (1999) Arthritis and Rheumatism 42, 641-649.
40. O, I., Kieber-Emmons, T., Otvos, L., and Blaszczyk-Thurin, M. (2000) Biochemical and Biophysical Research Communications 268, 106-111.
41. O, I., Otvos, L., Kieber-Emmons, T., and Blaszczyk-Thurin, M. (2002) Peptides 23, 999-1010.
42. Molenaar, T. J. M., Appeldoorn, C. C. M., de Haas, S. A. M., Michon, N. N., Bonnefoy, A., Hoylaerts, M. F., Pannekoek, H., van Berkel, T. J. C., Kuiper, J., and Biessen, E. A. L. (2002) Blood 100, 3570-3577.
43. Molenaar, T. J. M., Twisk, J., de Haas, S. A. M., Peterse, N., Vogelaar, B., van Leeuwen, S. H., Michon, I. N., van Berkel, T. J. C., Kuiper, J., and Biessen, E. A. L. (2003) Biochemical Pharmacology 66, 859-866.
44. Pereira, A., del Valle Onorato, M., and Sanz, C. (2003) British Journal of Haematology 120, 814-820.

We claim:

1. A composition comprising one or more selectin binding molecule covalently linked to a hydrophilic polymer.

2. The composition of claim 1 wherein the composition comprises more than one selectin binding molecule.

3. The composition of claim 2, wherein the composition comprises more than one copy of the same selectin binding molecule.

4. The composition of claim 2, wherein the composition comprises different selectin binding molecules.

5. The composition of claim 4, wherein the different selectin binding molecules have the same selectin specificity.

6. The composition of claim 4, wherein the different selectin binding molecules have different selectin specificities.

7. The composition of claim, 1 wherein the selectin binding molecule comprises a compound selected from the group consisting of the compounds presented in Table 1.

8. The composition of claim, 1 wherein the selectin binding molecule comprises a polypeptide.

9. The composition of claim, 8 wherein the polypeptide is selected from the group consisting of SEQ ID NOS. 1-12.

10. The composition of claim, 1 wherein the hydrophilic polymer comprises a polysaccharide.

11. The composition of claim, 10 wherein the polysaccharide comprises dextran.

12. The composition of claim 1 wherein the selectin binding molecules are clustered at one terminus of the hydrophilic polymer.

13. A pharmaceutical composition comprising the composition of claim 1 and a pharmaceutically acceptable carrier.

14. A method for treating inflammatory disorders comprising administering to a patient in need thereof an amount effective to treat the inflammatory disorder of the composition of claim 1.

15. The method of claim 14, wherein the inflammatory disorder is selected from the group consisting of septic shock, post-trauma multiple organ failure, ischemic reperfusion injury, transplant rejection, autoimmune disease, rheumatoid arthritis, psoriasis, inflammatory bowel disease, adult respiratory distress syndrome, tumor metastasis, and burns.


SEQUENCE LISTING (12 sequences)

SEQ ID NO: 1 - protein, 7 aa, Artificial sequence
  Synthetic peptide
IELLQAR

SEQ ID NO: 2 - protein, 13 aa, Artificial sequence
  Synthetic peptide
CDITWAQLWDLMK

SEQ ID NO: 3 - protein, 16 aa, Artificial sequence
  Synthetic peptide
KYDGDITWDQLWDLMK

SEQ ID NO: 4 - protein, 12 aa, Artificial sequence
  Synthetic peptide
DLWDWVVGKPAG

SEQ ID NO: 5 - protein, 8 aa, Artificial Sequence
  Synthetic peptide
DXXDXXVG

SEQ ID NO: 6 - protein, 5 aa, Artificial sequence
  Synthetic peptide
EWVDV

SEQ ID NO: 7 - protein, 5 aa, Artificial sequence
  Synthetic peptide
VVGXP

SEQ ID NO: 8 - protein, 6 aa, Artificial sequence
  Synthetic peptide
FVVGXP

SEQ ID NO: 9 - protein, 12 aa, Artificial sequence
  Synthetic peptide
DLWDFVVGKPAG

SEQ ID NO: 10 - protein, 15 aa, Artificial sequence
  Synthetic peptide
LVSVLDLEPLDAAWL

SEQ ID NO: 11 - protein, 19 aa, Artificial sequence
  Synthetic peptide
QATEYEYLDYDFLPETEPP

SEQ ID NO: 12 - protein, 13 aa, Artificial sequence
  Synthetic peptide
CDITWDQLWDLMK